(19) United States
(12) Patent Application Publication
(10) Pub. No.: US 2003/0229366 A1
(43) Pub. Date: Dec. 11, 2003

(54) Implantable lumen occluding devices and methods

(75) Inventors: John A. Reggie, Palo Alto, CA (US); Joshua Makower, Los Altos, CA (US); J. Christopher Flaherty, Topfield, MA (US)

Correspondence Address: Robert D. Buyan, Stout, Uxa, Buyan & Mullins, LLP, Suite 300, 4 Venture, Irvine, CA, 92618, US

(73) Assignee: TransVascular, Inc., Menlo Park, CA

(21) Appl. No.: 10/391,481

(22) Filed: Mar. 17, 2003

Related U.S. Application Data
(63) Continuation-in-part of application No. 09/117,515, filed on Jan. 11, 1999, now Pat. No. 6,579,311.
(63) A 371 of international application No. PCT/US97/01459, filed on Jan. 31, 1997.
(63) Continuation-in-part of application No. 08/730,327, filed on Oct. 11, 1996, now Pat. No. 6,190,353.
(63) Continuation-in-part of application No. 08/730,496, filed on Oct. 11, 1996, now Pat. No. 5,830,222.
(60) Provisional application No. 60/364,439, filed on Mar. 15, 2002.
(60) Provisional application No. 60/010,614, filed on Feb. 2, 1996.

(51) Int. Cl. A61F 2/06

(52) U.S. Cl. 606/158; 623/1.12

(57) ABSTRACT

Implantable embolic devices for occluding the lumens of blood vessels and other anatomical conduits comprising a generally tubular, radially expandable frame and a flexible occluder member attached to the frame. The flexible occluder member may be of generally tubular form having a closed end and an open end. The open end is attached to the frame and the closed end serves to substantially block the flow of blood through the lumen of the anatomical conduit. In some embodiments a small or self-sealing opening is formed in the closed end of the flexible occluder member such that a guidewire, catheter or other device may pass through such opening during delivery of the device and/or at some later time following implantation of the device.

### CROSS-REFERENCE TO RELATED APPLICATIONS

This application also claims priority to U.S. Provisional Patent Application Serial No. 60/364,439 filed on Mar. 15, 2002. This application is also a continuation-in-part of copending U.S. patent application Ser. No. 09/117,516 filed on Jan. 21, 1999 which a) is a national stage filing under 35 U.S.C. 371 of PCT/US97/01463 filed on Jan. 31, 1997, b) claims priority to U.S. Provisional Patent Application No. 60/101,614 filed on Feb. 2, 1996 and c) is a continuation-in-part of U.S. patent application Ser. No. 08/730,327 filed on Oct. 11, 1996, now abandoned and U.S. patent application Ser. No. 08/730,496 filed on Oct. 11, 1996 and now issued as U.S. Pat. No. 5,830,222.

### FIELD OF THE INVENTION

The present invention relates generally to medical devices and methods and more particularly to implantable devices for occluding the lumens of blood vessels or other luminal anatomical structures and their methods of use.

### BACKGROUND OF THE INVENTION

Implantable embolic devices are used to occlude the lumens of blood vessels or other anatomical conduits of the body. Such embolic devices have been used for a variety of therapeutic purposes. For example, certain procedures known as PICVA™ and PICAB™ are being developed by TransVascular, Inc. of Menlo Park, Calif. These procedures utilize native veins as in situ bypass conduits for diseased arteries. In such procedures, it is typically desirable to place at least one embolic blocker in the lumen of the vein into which arterial blood has been routed to in such procedures, including blocking of blood flow in veins into which arterial blood has been routed to facilitate the intended flow of arterial blood through the vein in a direction opposite normal venous flow. Examples of these PICVA™ and PICAB™ procedures are described in U.S. Pat. Nos. 5,830,222 (Makower), 6,068,638 (Makower), 6,190,353 (Makower et al.) and 6,302,875 (Makower et al.), which are expressly incorporated herein by reference.

Examples of some of the implantable embolic blockers of the prior art are described in U.S. Pat. Nos. 5,830,222 (Makower), 6,071,292 (Makower et al., 6,287,317 (Makower et al.) and 5,499,995 (Teirstein) as well as PCT International Publication No. Wo/97/270893 (Evard et al.), which are expressly incorporated herein by reference.

Although some of the embolic devices of the prior art may be useable to effectively block flow though some blood vessels or other body conduits, are remains a need in the art for the development of new implantable embolic devices and methods for catheter based, transluminal delivery and implantation of such devices.

### SUMMARY OF THE INVENTION

The present invention provides an implantable embolic device for blocking the flow of body fluid through an anatomical conduit that has a wall and a lumen (e.g., blood vessel, duct, passageway, respiratory passage, bronchus, lymphatic, iatrogenically created channel or opening, shunt, etc.). In general, the implantable embolic device comprises a generally tubular, radially expandable frame member and a flexible occluder member attached to the frame member. The flexible occluder member may be formed of any suitable material, such as expanded polytetrafluoroethylene (ePTFE), that is generally in the form of a tube having an open first end and a substantially closed second end. The open first end of the flexible occluder member is affixed (or otherwise held in abutment with) to the frame member. The device is initially disposed in a first radially collapsed configuration wherein it may be transluminally advanced into the lumen of the anatomical conduit in which it is to be implanted. Thereafter, the device is expandable to a second radially expanded configuration wherein it will engage the wall of the anatomical conduit such that the closed end of the flexible member will substantially block the flow of body fluid through the lumen of the anatomical conduit. The frame member may be self-expanding or pressure expandable and may be formed of any suitable material, such as metal or plastic. In a preferred embodiment the frame is formed of a nickel titanium alloy that is superelastic at normal body temperature of 37° C. In some embodiments, the flexible occluder member may have an opening (e.g., a small hole or self-sealing opening) formed in its closed end. A catheter, guidewire or other object may be passed through such opening. Where the opening is self-sealing, the opening will resume a substantially closed configuration after such catheter guidewire or other object is removed, such that no body fluid or no more than a clinically insignificant amount of body fluid will leak though such opening. In other embodiments the opening may simply be so small in size that it the amount of body fluid that leaks through such opening is not clinically significant or does not defeat the intended embolic function of the device. Also, in some embodiments, the flexible occluder member may cover a portion of the frame adjacent its first end while a portion of the frame adjacent its second end remains uncovered. Such partially covered embodiment of the device may be implanted in the lumen of a blood vessel or other body conduit such that pressure of body fluid distal to the first end of the frame is greater than the pressure of body fluid proximal to the second end of the frame. This serves to ensure that at least the uncovered portion of the frame will remain in firm frictional engagement even if the pressure of body fluid creates some gap or space between the covered portion of the frame and the adjacent wall of the anatomical conduit. Also, in self expanding embodiments, such partial covering of the frame will allow the uncovered portion of the frame to remain expandable without being constrained or restricted by the flexible covering.

Further in accordance with the invention, an embolic device of the foregoing character is mounted on a delivery catheter for catheter-based transluminal delivery and implantation of the device. The delivery catheter may comprise an outer tube having a wall and a lumen and an inner tube having a wall and a lumen, with the inner tube being disposed within the lumen of the outer tube. The embolic device is mounted on the outer tube while in its first radially collapsed configuration. For embodiments where the frame is pressure expandable, a generally cylindrical balloon or other radially expandable member may be positioned on the delivery catheter beneath the embolic device to effect radial expansion and implantation of the embolic device. For embodiments where the frame is self-expanding, the embolic device may be initially loaded into the lumen of the outer tube and advanced therefrom by a pusher element or other suitable ejection apparatus. Alternatively, for self-expanding embodiments, the embolic device may be mounted about the exterior of the outer tube and one or more constraining members (e.g., a retractable sheath, severable skin or covering, retractable clip(s)s, etc.) will radially constrain the embolic device, holding it in its first collapsed configuration until such time as it is desired to allow the device to radially expand in situ to its second radially expanded configuration. In embodiments where the closed end of the flexible occluder member has an opening formed therein, a distal portion of the delivery catheter's inner tube may initially extend through such opening. A guidewire or other elongate apparatus may extend through the lumen of the inner tube to a location distal of the embolic device. Also, radiographic contrast agent, medicaments or other substances may be injected through the lumen of the inner tube. Also, in embodiments where an opening is formed in the closed end of the flexible occluder member, the embolic device may be re-traversed subsequent to its implantation by advancing a guidewire, catheter or other elongate apparatus through the opening. This may allow for performance or therapeutic or diagnostic procedures at locations distal to the implanted embolic device without requiring removal of the embolic device.

Further objects and aspects of the present invention will become apparent to those of skill in the art upon reading and considering the detailed description and examples set forth herebelow.

### BRIEF DESCRIPTION OF THE DRAWINGS

FIG. 1 is a side view of one embodiment of an embolic device of the present invention.

FIGS. 2a-2f are a step-by-step showing of one example of a method for assembling the embolic device of FIG. 1.

FIGS. 3a-3d are a step-by-step showing of one example of a method for transluminal catheter based delivery of the embolic device shown in FIG. 1.

FIG. 3d is a perspective view of the embolic device of FIG. 1 implanted in the lumen of a blood vessel wherein the pressure of the blood distal to the device has created a gap between a covered portion of the device and the surrounding blood vessel wall while the uncovered portion of the device remains in abutting coaptation with the surrounding blood vessel wall.

### DETAILED DESCRIPTION AND EXAMPLES

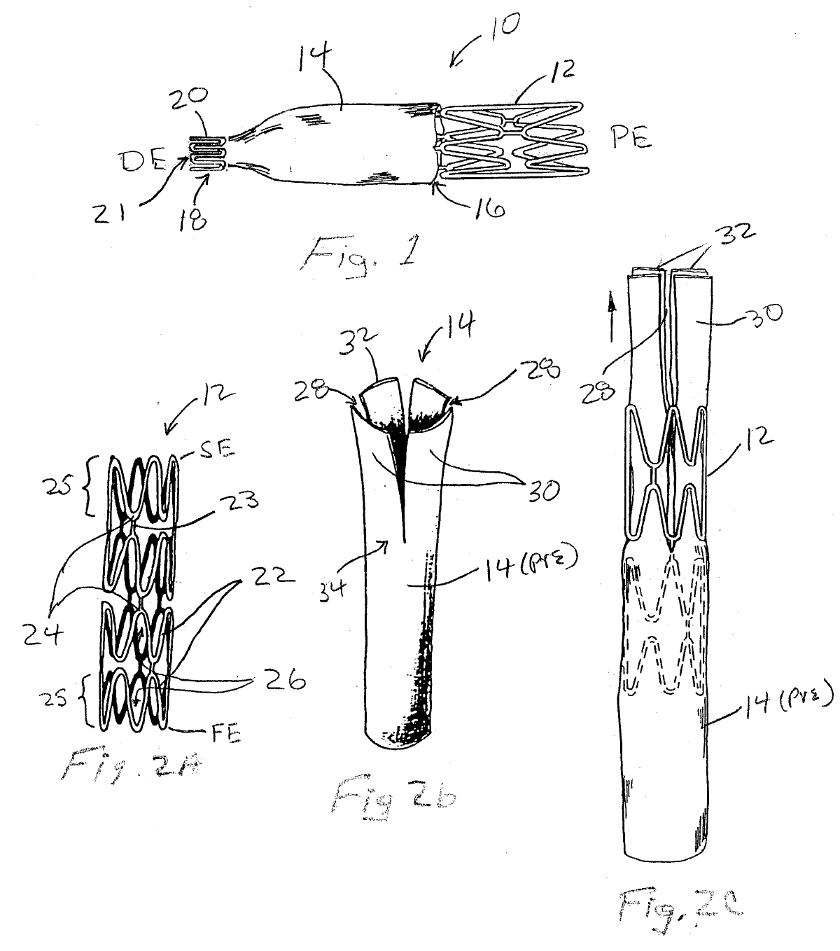

The following detailed description, and the accompanying drawings to which it refers, are provided describing and illustrating certain examples or specific embodiments of the invention only and not for the purpose of exhaustively describing all possible embodiments and examples of the invention. Thus, this detailed description does not in any way limit the scope of the inventions claimed in this patent application or in any patent(s) issuing form this or any related application. FIG. 1 shows one example of an embolic device 10 of the present invention. The embolic device 10 comprises a generally tubular frame member 12 and a flexible member 14. The device 10 has a proximal end PE and a distal end DE. The flexible member 14 is generally in the form of a tube having an open first end 16 and a substantially closed second end 18. The open first end 16 of the flexible member 14 is affixed to the frame member 12, as shown.

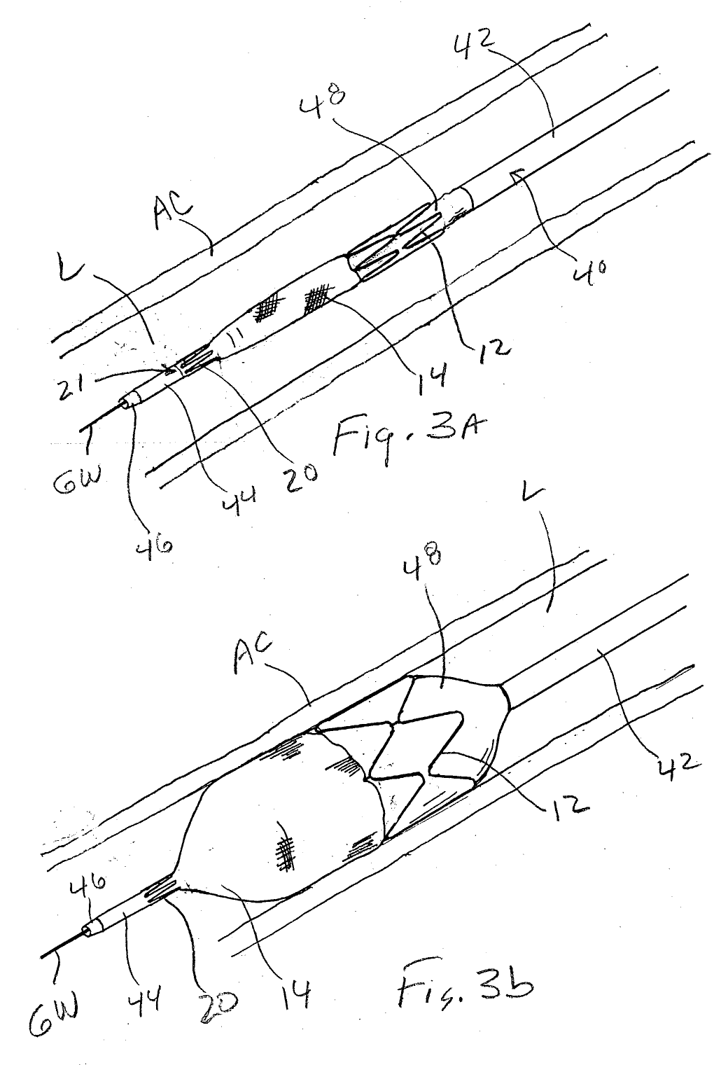

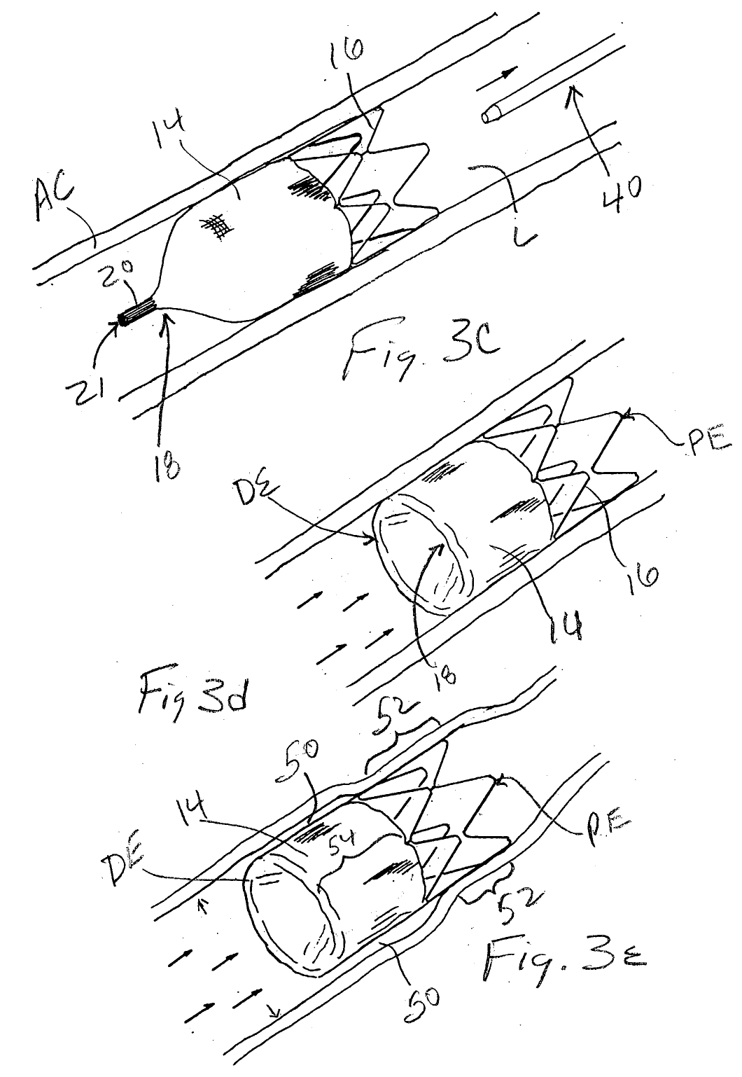

The embolic device 10 is initially disposable in a first configuration (se FIG. 3a and the description below) wherein it may be transluminally advanced into the lumen of said anatomical conduit and subsequently expandable to a second configuration (see FIG. 3b and the description below) wherein it will engage the wall of an anatomical conduit in which is it positioned. When so positioned in the lumen of the anatomical conduit, the closed end 18 of the flexible member 14 will substantially occlude or block the flow of body fluid through the lumen of the anatomical conduit.

The frame member 12 may be formed of any suitable radially expandable material such as a metal or plastic. In a presently preferred embodiment, the frame member 12 is formed of a stainless steal that is plastically deformable. Also, in the embodiment shown in the drawings, the frame member 12 comprises a plurality of zigzag rings 25 that are connected in alignment with one another by linking segments 23. Each zig-zag ring 25 of the frame 12 comprises a plurality of generally straight segments 22 connected to one another at angles so as to form apices 24 and troughs 26, as shown. In some embodiments the frame member 12 may be formed of resilient material that, when unconstrained, will self-expand from the first configuration (FIG. 3a) to the second configuration (FIG. 3b). Such self-expanding embodiments of the device 10 may be mounted on or in a delivery catheter that is constructed to constrain the device in its first configuration while it is being transluminally advanced into the lumen of the body conduit and to then to allow the operator to remove the constraint from the device 10, thereby allowing the device 10 to self-expand to its second configuration within the lumen of the anatomical conduit. In other embodiments, the frame member 12 may be formed of plastically deformable material may be expanded from its first configuration to its second configuration by exertion of outwardly directed radial force upon said frame member. Such pressure-expandable embodiments of the device 10 may be mounted on or in a delivery catheter that is equipped with a balloon or other radially expandable member useable to exert outwardly directed radial force upon the frame member 12 causing the device 10 to expand to its second configuration within the lumen of the anatomical conduit (see FIGS. 3a-3b and discussion set forth herebelow).

The embolic device shown in the drawings includes an optional self-sealing opening 21 formed in the closed end 18 of the flexible member 14. A compression band 20 is positioned about the distal end of the flexible member 14 to compress it to a closed configuration. A catheter, guidewire or other elongate apparatus may be advanced through the self-sealing opening 21 during delivery of the device or after the device has been implanted in the lumen of a body conduit. The compression band is preferably formed of elastic or superelastic material (e.g., a rubber band, elastic thread(s), superelastic NiTi alloy, etc.) In the particular example shown, the compression band 20 is formed of nickel titanium alloy that is superelastic at body temperature and is generally of a zig-zag shape, as shown. The compression band 20 will dilate as a catheter, guidewire or other elongate apparatus is advance though the self-sealing opening 21, with the flexible member 14 being firmly compressed therearound so as to deter leakage. When such catheter, guidewire or other elongate apparatus is subsequently removed from the self sealing opening, the compression band 20 will resiliently and/or elastically compress the opening 21 closed such that little or no body fluid will leak through such opening 21. Those of skill in the art will appreciate that, in some applications, some leakage of body fluid may be acceptable or even desirable. Thus, the compression band 20 may be constructed so as not to cause complete closure of the self sealing opening 21. In other embodiments, the self-sealing opening may be replaced by a small opening that is large enough to permit passage therethrough of a guidewire, catheter or other device but yet small enough to allow leakage of only a volume of body fluid that is not sufficiently large to defeat or substantially interfere with the intended clinical function of the device.

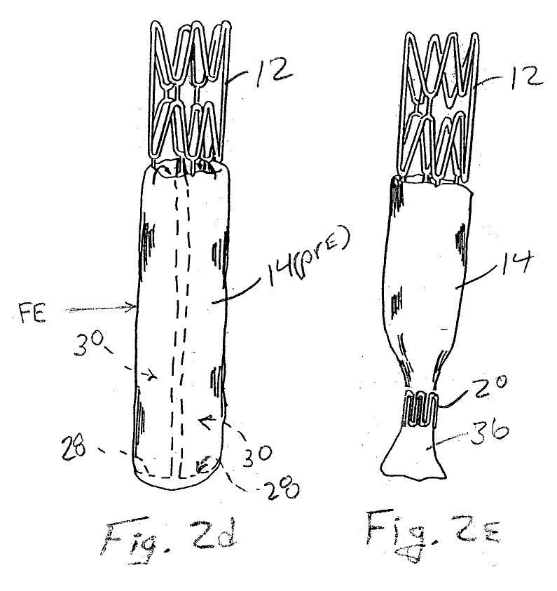

FIGS. 2a-2e show one example of a method for assembling the embolic device 10. As shown in FIG. 2b, a plurality of longitudinally oriented slits 28 are formed in one end of a tubular workpiece 14(pre) formed of flexible material such as ePTFE. The formation of these slits 28 creates a plurality of strips 30 at one end of the workpiece 14(pre), each such strip 30 having a free end 32 and an attached end 34. In the preferred embodiment, the slits 28 extend approximately one half the length of the tubular workpiece 14(pre). As shown in FIGS. 2c and 2d, approximately one half of the frame member 12 is then inserted into the lumen of the tubular workpiece 14(pre) and the free ends 32 of the strips 30 are passed though openings in the frame. The strips 30 are then doubled back through the lumen of the workpiece 14(pre) such that the free ends 32 of the strips 30 extend beyond the first end FE of the frame 12, as indicated by the dotted lines on FIG. 2d. Thereafter, the compression band 20 is positioned about the tubular workpiece 14(pre) at a location beyond the first end FE of the frame 12 such that the compression member will compress and anchor the strips 30 to the surrounding tubular body of the workpiece 14(pre). This results in formation of the closed end 18 and the self sealing opening 21. This also serves to soundly anchor the free ends 28 of the strips 30 such that the strips 30 do not pull back through the openings in the frame 12 and the open end 16 of the flexible member 14 is thereby affixed to the frame 12.

FIGS. 3a-3d show in step-by-step fashion a method for delivery and implantation of a pressure expandable embodiment of the embolic device 10. As shown in FIG. 3a, the embolic device 10 is initially mounted upon a delivery catheter 40. This delivery catheter 40 comprises an outer tube 42 which has a wall and a lumen extending longitudinally therethrough and an inner tube 44 which also has a wall and a lumen. The inner tube 44 is disposed within the lumen of the outer tube 42. A generally cylindrical balloon 48 is mounted about the outer surface of a portion of the outer tube 42 and the embolic device 10 is mounted over the balloon 48 in its second radially collapsed configuration (FIG. 3a). A distal portion of the inner tube 44 extends beyond the distal end of the outer tube 42 and through the self-sealing opening 21. A tapered region 46 may optionally be formed at the distal end of the inner tube 44 to facilitate dilation of the self-sealing opening 21 as the inner tube is advanced therethrough. Optionally, a guidewire GW or other apparatus may pass through the lumen of the inner tube 44 and out of its distal end. Also, radiographic contrast media or other substances may be injected through the lumen of the inner tube 44 before or after radial expansion of the embolic device 10. Also, it will be appreciated that a fluid may be placed in the lumen of the inner tube and a pressure transducer may be attached to permit monitoring or pressures within the lumen L of the anatomical conduit AC.

The delivery catheter 40 having the embolic device 10 mounted thereon in its collapsed configuration is advanced into the lumen L of the anatomical conduit AC in which the device is to be implanted. Radiographically visible markers may be formed on the delivery catheter 40 and/or embolic device 14 to enable the operator to verify that the embolic member is at the desired position of implantation. Thereafter, the balloon 48 is inflated so as to radially expand the embolic device, as shown in FIG. 3b. The expanded frame 12 frictionally engages the wall of the anatomical conduit AC. The balloon is then deflated and the delivery catheter 40 and any guidewire GW or other device is removed as shown in FIG. 3c. This causes the self sealing opening 21 to close and the closed end 18 of the flexible member substantially occludes the lumen L of the anatomical conduit AC.

As indicated in FIG. 3d, the pressure of body fluid within the lumen L of the anatomical conduit AC, distal to the implanted device 10, is greater than the pressure within the lumen L proximal to the implanted device. This pressure differential causes the closed end 18 of the flexible member to invert into the interior of the frame 12, as shown.

As illustrated in FIG. 3d, in some instances, the wall of the anatomical conduit AC distal to the device 10 may dilate due to increased pressure or for other reasons. Such dilation of the anatomical conduit wall may result in formation of a gap or space 50 between the distal portion 54 of the device 10 (which is covered by the flexible member) and the adjacent wall of the anatomical conduit. However, because the frame 12 in the proximal portion 52 of the device 10 is not covered by the flexible member 14, any body fluid that seeps from the gap 50 past the distal portion of the device 10 will pas through the openings in the uncovered frame 12 and will not result in disruption of the contact between the proximal portion 52 of the device 10 and the surrounding anatomical conduit wall. This helps to deter any migration or movement of the implanted device 10.

As will be appreciated from the above-set-forth description, the embolic device 10 and methods of the present invention may provide several advantages over the prior art. For example, the embolic device 10 of the present invention causes rapid or substantially instantaneous occlusion of the vessel lumen and does not rely on changes that must occur over time, as may be the case with other approaches like glues and implantable occlusion coils. Also, a single embolic device 10 serves to occlude a vessel lumen whereas a number of coils or multiple applications of glue could be required in some cases. Also, during routine use, the over-the-wire, balloon expandable version of this device 10 does not become “free-floating” in the blood stream. The physician retains control over the device 10 either via the delivery catheter 10 or guidewire, if deployed. This approach provides a high degree of control over position of the embolic device 10 and occupies only a short length of occluded vessel as opposed to certain types of occlusion coils that may create a mass several centimeters long within a blood vessel. Also, the device 10 provides permanent occlusion of the anatomical conduit and does not tend to recannalize overtime as may occur with some other occlusion techniques.

Although exemplary embodiments of the invention have been shown and described, many changes, modifications and substitutions may be made by those having ordinary skill in the art without necessarily departing from the spirit and scope of this invention. For example, elements, components or attributes of one embodiment or example may be combined with or may replace elements, components or attributes of another embodiment or example to whatever extent is possible without causing the embodiment or example so modified to become unuseable for its intended purpose. Accordingly, it is intended that all such additions, deletions, modifications and variations be included within the scope of the following claims. Also, although several illustrative examples of means for practicing the invention are described above, these examples are by no means exhaustive of all possible means for practicing the invention. The scope of the invention should therefore be determined with reference to the appended claims, along with the full range of equivalents to which those clams are entitled.

What is claimed is:

1. An implantable embolic member for blocking the flow of body fluid through an anatomical conduit that has a wall and a lumen, said device comprising:
a generally tubular frame member; and,
a flexible member generally in the form of a tube having an open first end and a substantially closed second end, the open first end of the flexible member being affixed to the frame member; the device being initially disposable in a first configuration wherein it may be transluminally advanced into the lumen of said anatomical conduit and subsequently expandable to a second configuration wherein it will engage the wall of the anatomical conduit such that the closed end of the flexible member will substantially block the flow of body fluid through the lumen of the anatomical conduit.

2. A device according to claim 1 wherein the generally tubular frame member comprises a mesh frame.

3. A device according to claim 1 wherein the generally tubular frame member is formed at least partially of a material that is superelastic at body temperature.

4. A device according to claim 3 wherein the generally tubular frame member is formed at least partially of nickel-titanium alloy.

5. A device according to claim 1 wherein the generally tubular frame member is formed of resilient material that, when unconstrained, will self-expand from the first configuration to the second configuration.

6. A system comprising a device according to claim 5 further in combination with a delivery catheter, said device being mounted on or in the delivery catheter, said delivery catheter being constructed to constrain the device in its first configuration as it is transluminally advanced into the lumen of the body conduit, at which time the operator may cause the constraint to be removed from the device, thereby allowing the device to self-expand to its second configuration within the lumen of the anatomical conduit.

7. A device according to claim 1 wherein the generally tubular frame member is formed of plastically deformable material that may be expanded from its first configuration to its second configuration by exertion of outwardly directed radial force upon said frame member.

8. A system comprising a device according to claim 7 further in combination with a delivery catheter, said delivery catheter comprising an elongate catheter body having a radially expandable member thereon and said device being mounted about said radially expandable member while in its first configuration as it is advanced into the lumen of the body conduit, at which time the operator may cause the radially expandable member to radially expand, thereby exerting outwardly directed radial pressure on the frame member and causing the device to expand to its second configuration within the lumen of the anatomical conduit.

9. A device according to claim 1 wherein the substantially closed end of the flexible member is biased to its substantially closed configuration by a compression band positioned about the flexible member.

10. A device according to claim 9 wherein the compression band comprises a band having a zig zag configuration.

11. A device according to claim 9 wherein the compression band is at least partially formed of a material that is elastic or superelastic.

12. A device according to claim 9 wherein the elastic band member is formed at least partially of nickel-titanium alloy.

13. A system comprising a device according to claim 1 further in combination with a delivery catheter, said delivery catheter comprising an outer tube having a wall and a lumen and an inner tube having a wall and a lumen, the inner tube being disposed within the lumen of the outer tube, the device being mounted on the outer tube, the closed end of the flexible member having an opening formed therein and a distal portion of the inner tube extending through said opening.

14. A system according to claim 13 further comprising a self-sealing component for causing the opening to close when the inner tube member is removed from said opening.

15. A system according to claim 14 wherein the self-sealing component causes the opening to substantially close when the inner tube of the delivery catheter is removed such that it no longer extends through said opening.

16. A system according to claim 13 wherein the self-sealing component comprises a compression band positioned about the flexible member so as to inwardly compress the flexible member.

17. A device according to claim 1 wherein the frame member comprises a plurality of zig-zag rings in alignment with one another and a plurality of linking segments connecting said zig-zag rings to one another.

18. A device according to claim 17 wherein the zig-zag rings comprise generally straight segments connected to one another at angles so as to form apices and troughs and wherein the linking segments extend between apices of adjacent zig-zag rings.

19. A device according to claim 1 wherein the frame member has openings formed therein and wherein the device is assembled by a process comprising the steps of:
i) forming a plurality of partial longitudinal slits in the open end of the flexible member so as to create a plurality of strips having free ends;
ii) passing the free ends of the strips through openings in the frame; and
iii) securing the free ends of the strips to prevent them from being pulled back through the openings in the frame, thereby attaching the flexible member to the frame.

20. A device according to claim 19 wherein Step i of the assembly process comprises
obtaining a workpiece formed of generally tubular flexible material, said workpiece having a hollow lumen and first and second open ends; and
forming slits that extend from one end of the workpiece to its approximate longitudinal midpoint so as to create a plurality of strips having free ends;
and wherein Step iii comprises;
passing the of the free ends of the strips through openings in the frame;
doubling the strips back through the hollow lumen of the workpiece; and,
placing a compression band around the workpiece such that the compression band collapses the tubular workpiece to form said closed end and anchors the strips so that the free ends of the strips do not pull back through the openings in the frame.

21. A device according to claim 20 wherein the method by which the assembly process further comprises the step of trimming away any residual flexible material distal to the compression band.

22. A device according to claim 20 wherein the compression band comprises a self-collapsing ring.

23. A device according to claim 22 wherein the self collapsing ring is generally of a zig-zag configuration.

24. A device according to claim 22 wherein the self-collapsing ring is formed at least partially of nickel titanium alloy.

25. A device according to claim 1 wherein there is a self-sealing opening formed in the closed end of the flexible member, said self-sealing opening being biased to a substantially closed configuration such that it will remain substantially closed when no object is inserted through said self-sealing opening but will dilate to an open configuration when an object is inserted therethrough.

26. A device according to claim 25 wherein, after the device has been implanted in the lumen of an anatomical conduit, the flexible member may be traversed in situ by advancement of a catheter or other object through the self-sealing opening.

27. A device according to claim 1 wherein the flexible member is formed at least partially of ePTFE.

28. A device according to claim 1 wherein the frame has a first end and a second end, a portion of the frame adjacent its first end being covered by the flexible member and a portion of the frame adjacent its second end not being covered by the flexible member.

29. A device according to claim 28 wherein the device is oriented in the lumen of an anatomical conduit such that the pressure of body fluid distal to the first end of the frame is greater than the pressure of body fluid proximal to the second end of the frame.

30. A method for blocking the flow of body fluid through an anatomical conduit that has a wall and a lumen, said method comprising the steps of:
A. providing a lumen occluding device that comprises i) a generally tubular frame member and ii) a flexible member comprising a tube having an open first end and a substantially closed second end, the open first end of the flexible member being affixed to the frame member, said lumen occluding device being initially disposable in a first configuration wherein it may be transluminally advanced into the lumen of said anatomical conduit and subsequently expandable to a second configuration wherein it will engage the wall of the anatomical conduit such that the closed end of the flexible member will substantially block the flow of body fluid through the lumen of the anatomical conduit;
B. positioning the lumen occluding device within the lumen of the 14 anatomical conduit while the device is in its first configuration; and, thereafter
C. causing the lumen occluding device to expand to its second configuration such that the lumen occluding device frictionally engages the wall of the anatomical conduit and the closed end of the flexible member substantially blocks the flow of body fluid through the lumen of the anatomical conduit.

31. A method according to claim 30 wherein the device provided in Step A is provided in combination with a delivery catheter, said delivery catheter comprising an outer tube having a wall and a lumen and an inner tube having a wall and a lumen, the inner tube being disposed within the lumen of the outer tube, the device being mounted on the outer tube, the closed end of the flexible member having an opening formed therein and a distal portion of the inner tube extending through said opening.

32. A method according to claim 31 wherein the device provided in Step A further comprises a guidewire extending through the inner tube of the delivery catheter.

33. A method according to claim 32 wherein Step B comprises:
advancing the guidewire into the lumen of the anatomical conduit;
advancing the delivery catheter over the guidewire;
causing the lumen occluding device to radially expand to its second configuration;
withdrawing the delivery catheter and guidewire from the lumen of the anatomical conduit such that the radially expanded lumen occluding device remains in place and the self sealing opening assumed its substantially closed configuration as the inner tube and guidewire are removed therefrom.

34. A method according to claim 30 wherein the device is oriented within the lumen of the anatomical conduit such that pressure exerted by the flow of body fluid will cause the flexible member to invert within the radially expanded frame member.

35. A method according to claim 30 wherein the device provided in Step A has a self-sealing opening formed in the closed end of the flexible member and wherein the method further comprising the step of:
D. re-traversing the lumen occluding device after completion of Step C by inserting an object through the self sealing opening.

36. A method according to claim 35 wherein Step D comprises inserting a catheter through the self sealing opening.

37. A method according to claim 36 further comprising the step of:
E. using the catheter that has been inserted through the self sealing opening to perform a therapeutic or diagnostic procedure.

38. A method according to claim 30 wherein the frame member of the device provided in Step A has a first end and a second end, a portion of the frame adjacent its first end being covered by the flexible member and a portion of the frame adjacent its second end not being covered by the flexible member, and wherein Step B comprises:
positioning the device within the lumen of an anatomical conduit such that the pressure of body fluid distal to the first end of the frame is greater than the pressure of body fluid proximal to the second end of the frame.
